(19) United States
(12) Patent Application Publication
(10) Pub. No.: US 2006/0035888 A1
(43) Pub. Date: Feb. 16, 2006

(54) Memantine as adjunctive treatment to atypical antipsychotic in schizophrenia patients

(75) Inventors: Jeffrey Jonas, Hoboken, NJ (US); Allyson Gage, West New York, NJ (US)

Correspondence Address: DARBY & DARBY P.C., P. O. BOX 5257, NEW YORK, NY, 10150-5257, US

(73) Assignee: Forest Laboratories, Inc., New York, NY (US)

(21) Appl. No.: 11/178,153

(22) Filed: Jul. 7, 2005

Related U.S. Application Data
(60) Provisional application No. 60/586,553, filed on Jul. 9, 2004.

(51) Int. Cl. A61K 31/551; A61K 31/13; A61K 31/519

(52) U.S. Cl. 514/220; 514/259.41; 514/662

(57) ABSTRACT

The present invention provides a method for treating schizophrenia in a patient in need thereof, the method comprising administering to the patient a therapeutically effective amount of memantine, or a pharmaceutically acceptable salt thereof, and a therapeutically effective amount an atypical antipsychotic. The method of the present invention embodies both the co-administration of memantine with an atypical antipsychotic, and the use of memantine as an adjunctive treatment to treatment with an atypical antipsychotic.

This application claims the benefit of U.S. Provisional Patent Application No. 60/586,553, filed Jul. 9, 2004, which is hereby incorporated by reference in its entirety.

### FIELD OF THE INVENTION

The present invention provides a method for treating schizophrenia in a patient in need thereof, the method comprising administering to the patient a therapeutically effective amount of memantine, or a pharmaceutically acceptable salt thereof, and a therapeutically effective amount of at least one atypical antipsychotic.

### BACKGROUND OF THE INVENTION

The worldwide prevalence of schizophrenia is reported up to 1.5% with an annual incidence of 5 per 10,000 individuals. Diagnostic and Statistical Manual of Mental Disorders, 4^{th} Ed., revised 1994. The cardinal symptoms of schizophrenia fall into three domains—positive, such as delusions and hallucinations, negative, such as lack of drive and social withdrawal, and cognitive, such as problems with attention and memory. Current guidelines recommend atypical antipsychotics, including risperidone, olanzapine, quetiapine, ziprasidone, and aripiprazole, as first-line treatment for schizophrenia. These agents reduce the positive symptoms of psychosis similarly to typical antipsychotics, but with a more favorable side effect profile, including a lower incidence of extrapyramidal symptoms.

Based on dopamine hyperactivity dysfunction, both typical and atypical antipsychotics function via blockade of the dopamine receptor, particularly the D2 subtype receptor, to reduce positive symptoms, one of the three symptom domains of schizophrenia. The atypical antipsychotics have the added benefit of impacting some negative symptoms and possibly cognitive symptoms which has been attributed to serotonin receptor blockade.

However, the atypical antipsychotics still may take as long as 16 or more weeks to produce a response, and even with prolonged treatment are unlikely to result in greater than 50% improvement in symptoms with up to 40% of patients not responding at all. This has led to the common clinical practice of experimental use of high atypical doses, antipsychotic polypharmacy, and augmentation with other psychotropic drugs. Diagnostic and Statistical Manual of Mental Disorders, 4^{th} Ed., revised 1994; Zink M, Henn F A, Thome J. “Combination of amisulpride and olanzapine in treatment-resistant schizophrenic psychoses.” European Psychiatry 2003; 19:56-58.

Studies have shown that all domains of cognition, including attention, executive function, secondary (storage) memory, working memory, and semantic memory, may be affected in patients with schizophrenia. This is important as cognitive symptoms predict functional outcomes such as social function, school and work function, and activities of daily living, more so than positive or negative symptoms. Diagnostic and Statistical Manual of Mental Disorders, 4^{tb} Ed., revised 1994. While 15% of patients test within the normal range, approximately 35% of patients experience progressive cognitive decline, with some reaching an Alzheimer's type dementia. Krystal J, et al. “Therapeutic implications of the NMDA receptor antagonist model psychosis.” Program and abstracts of the 16th European College of Neuropsychopharmacology Congress; Sep. 20-24, 2003; Prague, Czech Republic. To date, standard antipsychotic drug regimens do not fully address the impact of cognitive symptoms associated with schizophrenia. In addition, anticholinergic medications used to treat extrapyramidal side effects (EPS) associated with antipsychotic drugs are thought to worsen components of cognition. Therefore there is a need in the art for adjunctive treatments to atypical antipsychotics in schizophrenia that fully address the impact of cognitive symptoms associated with the disease.

The present inventors have discovered that the systemically-active uncompetitive NMDA receptor, memantine (1-amino-3,5-dimethyladamantane), or a pharmaceutically acceptable salt thereof, may be used in combination with at least one antipsychotic, or as an adjunctive treatment to treatment with at least one antipsychotic to treat schizophrenia patients. Memantine, disclosed in U.S. Pat. Nos. 4,122,193; 4,273,774; and 5,061,703, all of which are hereby incorporated by reference, is currently available in the US and in over 42 countries worldwide. It is approved for the treatment of moderate to severe Alzheimer's disease (AD) in the United States at a dose of up to 20 mg/day (10 mg BID), however, its use in the treatment of schizophrenia has not been previously reported.

### SUMMARY OF THE INVENTION

In one embodiment present invention provides a method for treating schizophrenia in a patient in need thereof, the method comprising administering to the patient a therapeutically effective amount of memantine, or a pharmaceutically acceptable salt thereof, and a therapeutically effective amount of at least one antipsychotic. In a preferred embodiment the antipsychotic is an atypical antipsychotic and still more preferably an atypical antipsychotic selected from the group consisting of olanzapine, clozapine, risperidone, sertindole, quetiapine, ziprasidone, surmontill and aripiprazole. In another embodiment of the invention, the antipsychotic may be a typical antipsychotic.

The present invention provides a method for treating schizophrenia in a patient in need thereof, wherein the treatment comprises administering memantine, or a pharmaceutically acceptable salt thereof, as an adjunctive treatment where the patient is being treated with at least one atypical antipsychotic. In another embodiment, the present invention provides a method for treating schizophrenia in a patient in need thereof, wherein memantine, or a pharmaceutically acceptable salt thereof, and at least one atypical antipsychotic are co-administered as separate dosage forms or as a unitary dosage form.

In one embodiment of the present invention, memantine, or a pharmaceutically acceptable salt thereof, is administered to a patient in a range of from about 2.5 to about 100 mg/day, more preferably in a range of from about 5 to about 80 mg/day, and still more preferably in a range of from about 5 to about 20 mg/day.

The present invention also provides a method for treating schizophrenia in a patient in need thereof, wherein memantine, or a pharmaceutically acceptable salt thereof, is administered orally as a liquid, or in a tablet or bead form. In other embodiments memantine may be administered as immediate or modified release tablets or beads.

In another embodiment the present invention provides a method for treating at least one sign or symptom of schizophrenia in a patient in need thereof, the method comprising administering to the patient a therapeutically effective amount of memantine, or a pharmaceutically acceptable salt thereof, and a therapeutically effective amount of at least one atypical antipsychotic, wherein the sign or symptom is selected from the group consisting of delusions, hallucinations, disorganized speech, catatonic behavior, affective flattening, alogia and avolition.

### DETAILED DESCRIPTION

In accordance with the present invention, a method for adjunctive treatment to atypical antipsychotics is provided. Specifically, a method of treatment is provided for schizophrenia patients who are residually symptomatic by administering memantine, or one of its pharmaceutically acceptable salts, preferably its HCl salt to a human in need thereof.

In the present invention, the method of treatment includes a therapeutically effective dose of memantine, or one of its pharmaceutically acceptable salts, in an immediate release or modified release formulation. Preferred formulations of memantine include oral tablets, beads, and liquid formulations.

### Memantine

Memantine (1-amino-3,5-dimethyladamantane), which is an analog of 1-amino-cyclohexane (disclosed, e.g., U.S. Pat. Nos. 4,122,193; 4,273,774; 5,061,703; and 5,614,560), is a systemically-active uncompetitive NMDA receptor antagonist having low to moderate affinity for the receptor and strong voltage dependency and rapid blocking/unblocking kinetics. These pharmacological features allow memantine to block sustained activation of the receptor under pathological conditions and to rapidly leave the NMDA channel during normal physiological activation of the channel. Memantine and pharmaceutically acceptable salts thereof (e.g., the HCl salt, MW 215.77) is approved in the U.S. for treatment of Alzheimer's disease, and is currently approved outside the United States as an oral formulation for both Alzheimer's and Parkinson's Disease. Memantine has also been suggested to be useful in the treatment of AIDS dementia (U.S. Pat. No. 5,506,231), neuropathic pain (U.S. Pat. No. 5,334,618), and cerebral ischemia (U.S. Pat. No. 5,061,703).

According to the invention, memantine may be used in the form of a free base or a pharmaceutically acceptable salt. The synthesis of memantine free base may include the following steps: chlorination of 1,3-dimethyladamantan to 1-chlor-3,5-dimethyladamantan; and introduction of the acetyl group by Ritter-reaction with acetonitril/sulfuric acid to obtain the product 1-acetylamino-3,5-dimethyladamantan. This product together with sodium hydroxide and butanol are heated under reflux until total hydrolysis is achieved. After cooling down to room temperature the mixture is mixed with water. The aqueous layer is separated and discarded. The organic layer contains memantine free base in butanol. Memantine free base may also be obtained from memantine by hydrochloride, adding aqueous sodium hydroxide, extracting with toluol, washing with water and evaporating most of the toluol. The result will be memantine free base in toluol.

Suitable salts of the compound include, but are not limited to, acid addition salts, such as those made with hydrochloric, methylsulfonic, hydrobromic, hydroiodic, perchloric, sulfuric, nitric, phosphoric, acetic, propionic, glycolic, lactic pyruvic, malonic, succinic, maleic, fumaric, maleic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. In a preferred embodiment, the salt is memantine hydrochloride (C₁₂H₂₁N.HCl, MW 215.77). The term “salts” can also include addition salts of free acids or free bases. All of these salts (or other similar salts) may be prepared by conventional means. All such salts are acceptable provided that they are non-toxic and do not substantially interfere with the desired pharmacological activity.

In addition, it is possible to use any salts and free base form of memantine (collectively referred to as memantine), including polymorphs, hydrates and solvates as well as amorphous forms of memantine.

In a preferred embodiment of the invention, memantine is provided as its hydrochloride salt.

### Antipsychotics

In one embodiment of the present invention, memantine is administered as an adjunctive treatment to one or more antipsychotics. In another embodiment of the invention, memantine may be co-administered as a combination therapy with one or more antipsychotics.

In the present invention, the antipsychotic may be atypical or typical. Preferably, the antipsychotic is an atypical antipsychotic. Atypical antipsychotics offer several clinical benefits over conventional antipsychotics, including for example, superior side effect profiles, particularly with regard to extrapyramidal side effects (EPS). Atypical antipsychotics typically differ from typical antipsychotics in their “limbic-specific” dopamine type 2 (D2)-receptor binding. Atypical antipsychotics, also display a high ratio of serotonin type 2 (5-HT2)-receptor binding to D2 binding. Atypical antipsychotics have high affinity for the 5-HT2-receptor and function as antagonists of serotonin for the 5-HT2-receptor. See Beasley, et al., Neuropsychopharmacology 14:111-123, (1996).

Examples of atypical antipsychotics include, but are not limited to:

Olanzapine, 2-methyl-4-(4-methyl-1-piperazinyl)-10H-thieno[2,3-b][1,5]benzodiazepine, disclosed in U.S. Pat. No. 5,229,382, (Commercially available from Eli Lilly, Indianapolis, Ind. under the tradename Zyprexa®) which is hereby incorporated by reference, as being useful for the treatment of schizophrenia, schizophreniform disorder, acute mania, mild anxiety states, and psychosis.

Clozapine, 8-chloro-11-(4-methyl-1-piperazinyl)-5H-dibenzo[b,e][1,4]-diazepine, (Commercially available from Mylan Pharmaceuticals, Morgantown, W. Va. under the tradename Mylan®) disclosed in U.S. Pat. No. 3,539,573, which is herein incorporated by reference. Clinical efficacy of Clozapine in the treatment of schizophrenia has previously been disclosed. Hanes, et al., Psychopharmacol. Bull., 24, 62 (1988);

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, (Commercially available from Janssen under the tradename Risperdal®) and its use in the treatment of psychotic diseases are disclosed in U.S. Pat. No. 4,804,663, which is herein incorporated by reference.

Sertindole, 1-[2-[4-[5-chloro-1-(4-fluorophenyl)-1H-indol-3-yl]-1-piperidinyl]ethyl]imidazolidin-2-one, is described in U.S. Pat. No. 4,710,500. Its use in the treatment of schizophrenia is described in U.S. Pat. Nos. 5,112,838 and 5,238,945. U.S. Pat. Nos. 4,710,500; 5,112,838; and 5,238,945 are herein incorporated by reference in their entirety;

Quetiapine, 5-[2-(4-dibenzo[b,f][1,4]thiazepin-11-yl-1-piperazinyl)-ethoxy]ethanol, (Commercially available from Astra Zeneca, Wilmington, Del. under the tradename Seroquel®) its activity in assays which demonstrate utility in the treatment of schizophrenia are disclosed in U.S. Pat. No. 4,879,288, which is herein incorporated by reference;

Aripiprazole, 7-[4-[4-(2,3-dichlorophenyl)-1-piperazinyl]butoxy]-3,4-di-hydrocarbostyril, (Commercially available from Bristol-Meyers Squibb Co., Princeton, N.J. under the tradename Abilify®) is disclosed in U.S. Pat. Nos. 4,734,416 and 5,006,528, which hereby are incorporated by reference;

Ziprasidone, 5-[2-[4-(1,2-benzoisothiazol-3-yl)-1-piperazinyl]ethyl]-6-chloro-1,3-dihydro-2H-indol-2-one, (Commercially available from Pfizer Inc., New York, N.Y. under the tradename Geodon®) is disclosed in U.S. Pat. Nos. 4,831,031 and 5,312,925 and its activity in assays which demonstrate utility in the treatment of schizophrenia are described in U.S. Pat. No. 4,831,031, all of which are herein incorporated by reference; and

Surmontil (trimipramine maleate), 5-(3-dimethylamino-2-methylpropyl)-10,11-dihydro-5H-dibenz (b,f) azepine acid maleate (Commercially available from Odyssey Pharmaceuticals, Inc, North Hanover, N.J. under the tradename Surmotil®).

In other embodiments of the invention, the antipsychotic may be a typical antipsychotic. Possible typical antipsychotics include but are not limited to: acepromazine, benperidol, bromazepam, bromperidol, chlorpromazine, chlorprothixene, clotiapine, cyamemazine, diazepam, dixyrazine, droperidol, flupentixol, fluphenazine, fluspirilene, haloperidol, heptaminol, isopropamide iodide, levomepromazine, levosulpiride, loxapine, melperone, mesoridazine, molindone, oxypertine, oxyprothepine, penfluridol, perazine, periciazine, perphenazine, pimozide, pipamperone, pipotiazine, prochlorperazine, promazine, promethazine, prothipendyl, pyridoxine, sulpiride, sultopride, tetrabenazine, thioproperazine, thioridazine, tiapride, tiotixene, trifluoperazine, triflupromazine, trihexyphenidyl, and zuclopenthixol.

### Formulations

In conjunction with the methods of the present invention, the pharmaceutical compositions comprise a therapeutically effective amount of memantine, or one of its pharmaceutically acceptable salts may further comprise a carrier or excipient (all pharmaceutically acceptable).

According to the present invention, the dosage form of memantine may be a solid, semisolid or liquid formulation (see Remington's Pharmaceutical Sciences, Mack 5 Publishing Co., Easton, Pa.) according to the following description. Compositions for oral administration include capsules, tablets, chewable tablets, melt fast dissolvable tablets, dispersible powders, granules, beads, liquids, syrups, elixirs and suspensions. These compositions can contain one or more conventional adjuvants, such as sweetening agents, flavoring agents, coloring agents and preserving agents.

For oral administration in the form of a tablet or capsule, memantine can be combined with a non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

The orally administered medicaments may also be administered in the form of a time-controlled release vehicle, including diffusion-controlled systems, osmotic devices, dissolution-controlled matrices, and erodible/degradable matrices.

Memantine is commercially available as the hydrochloride salt (Commercially available from Forest Laboratories under the tradename Namenda™) in 5 or 10 mg film-coated tablets. The tablets can be coated by methods well known in the art. The cores may also be coated with a concentrated sugar solution which may contain e.g., gum arabic, gelatine, talcum, titanium dioxide, and the like. Alternatively, the tablets can be coated with a polymer known to a person skilled in the art, wherein the polymer is dissolved in a readily volatile organic solvent or mixture of organic solvents. Tablets can contain the active ingredients in a mixture with conventional pharmaceutically acceptable excipients. These include inert carriers, such as calcium carbonate, sodium carbonate, lactose, and talc; granulating and disintegrating agents, such as starch and alginic acid; binding agents such as starch, gelatin acacia; and lubricating agents, such as magnesium stearate, stearic acid and talc. Tablets may be uncoated or coated by known techniques to delay disintegration and absorption in the gastrointestinal tract thereby providing a sustained action over a longer period of time.

In specific embodiments, memantine is formulated in to immediate-release (IR) and/or modified-release (MR) tablets. Immediate release solid dosage forms permit the release of most or all of the active ingredient over a short period of time, such as 60 minutes or less, and make rapid absorption of the drug possible so as to decrease the time to onset of relief when the dosage form is administered to a patient. Immediate release formulations are disclosed in U.S. patent application Ser. No. 11/155,319, filed Jun. 16, 2005, the disclosure of which is incorporated herein by reference in its entirety.

Modified release solid oral dosage forms permit the sustained release of the active ingredient over an extended period of time in an effort to maintain therapeutically effective plasma levels over similarly extended time intervals and/or to modify other pharmacokinetic properties of the active ingredient, in part to necessitate fewer daily administrations. Modified release formulations are disclosed in U.S. patent application Ser. No. 11/155,330, filed Jun. 16, 2005, the disclosure of which is incorporated herein by reference in its entirety.

For the formulation of soft gelatin capsules, the active substances may be admixed with, e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatin capsules. Capsules may contain the active ingredients alone or an admixture with an inert solid carrier, such as calcium carbonate, calcium phosphate or kaolin. Similarly, suspensions, syrups and elixirs may contain the active ingredients in mixture with any of the conventional excipients utilized in the preparation of such compositions. This includes suspending agents such as methylcellulose, tragacanth and sodium alginate; wetting agents such as lecithin, polyoxyethylene stearate or polyoxyethylene sorbitan monoleate; and preservatives.

The compositions of the invention can be also introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publication Nos. WO 95/11010 and WO 93/07861, all of which are hereby incorporated by reference). Biocompatible polymers useful in achieving controlled release of a drug, include for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

Memantine-coated non-pareil beads or seeds are also contemplated for use according to the present invention (see Huang et al., (2002) Drug Dev Ind Pharm. 28(5):593-9; and Ganesan et al., (2003) Boll Chim Farm. 142(7):290-4). Bead formulations are disclosed in U.S. Provisional Patent Application No. 60/691,512, filed Jun. 16, 2005, the disclosure of which is incorporated herein by reference in its entirety. Memantine bead formulations for use in the present invention may be either immediate or modified release beads, or a combination thereof.

Formulation of memantine in semi-solid or liquid form is within the skill of the art, as the active ingredient is highly soluble in aqueous media. Usually the active substance, i.e., memantine, will constitute between 0.1 and 99% by weight of the formulation, more specifically between 0.5 and 20% by weight for formulations intended for injection and between 0.2 and 50% by weight for formulations suitable for oral administration. In another embodiment of the invention, memantine is formulated in an oral, liquid formulation. A liquid formulation for oral administration is disclosed in U.S. Provisional Application No. 60/517,981, filed Nov. 5, 2003 and PCT Application No. PCT/US2004/037026, filed Nov. 5, 2004, the disclosures of which are incorporated herein by reference in their entirety. Liquid preparations for oral administration can take the form of, for example, solutions, syrups, emulsions or suspensions, or they can be presented as a dry product for reconstitution with water or other suitable vehicle before use. Preparations for oral administration can also be suitably formulated to give controlled or postponed release of the active compound. A particular example of an oral time-controlled release pharmaceutical formulation is described in U.S. Pat. No. 5,366,738, which is hereby incorporated by reference. In a preferred embodiment for the administration to pediatric subjects, memantine is formulated as a flavored liquid, e.g., peppermint flavor.

For oral administration in liquid form, memantine, or one of its pharmaceutically acceptable salts, can be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms. For example, solutions may contain from about 0.2% to about 20% by weight of memantine, with the balance being sugar and mixture of ethanol, water, glycerol and propylene glycol. Optionally such liquid formulations may contain coloring agents, flavoring agents, saccharine and carboxymethyl-cellulose as a thickening agent or other excipients known to a person skilled in the art. In one embodiment, a therapeutically effective amount of memantine is administered in an oral solution containing a preservative, a sweetener, a solubilizer, and a solvent. The present oral solution may include one or more buffers, flavorings, or additional excipients. In a further preferred embodiment, a peppermint or other flavoring is added to the oral liquid memantine formulation.

### Modes of Administration

The formulations of the invention are preferably delivered orally. Other modes of administration may include administration parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion.

Solutions for parenteral applications by injection can be prepared in an aqueous solution of a water-soluble pharmaceutically acceptable salt of the active substances, preferably in a concentration of from about 0.5% to about 10% by weight. These solutions may also contain stabilizing agents and/or buffering agents and may conveniently be provided in various dosage unit ampoules. Formulations for injection can be presented in unit dosage form, e.g., in ampoules or in multi-dose containers, with an added preservative. Alternatively, the active ingredient can be in powder form for reconstitution with a suitable vehicle, e.g., sterile pyrogen-free water, before use. For parental administration, the rate of infusion must be carefully controlled due to the relatively long half-life in the blood stream.

For administration by inhalation, can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Dosage units for rectal application can be solutions or suspensions or can be prepared in the form of suppositories or retention enemas in a mixture with a neutral fatty base, or gelatin rectal capsules comprising the active substances in admixture with vegetable oil or paraffin oil.

The invention also provides a pharmaceutical pack or kit comprising one or more containers containing memantine and, optionally, more of the antipsychotic ingredients. In a specific embodiment, memantine is provided as an oral solution (2 mg/ml) for administration with the use of a 2 teaspoon capacity syringe (dosage KORC®). Each oral syringe has blue hatch marks for measurement, with lines on the right side of the syringe (tip down) representing teaspoon units, and those on the left representing ml units.

### Dosages

The active ingredients of the present invention (e.g., memantine, antipsychotic) can be formulated for once-a-day administration or twice-a-day administration.

Preferably, the optimal therapeutically effective amount should be determined experimentally, taking into consideration the exact mode of administration, from in which the drug is administered, the indication toward which the administration is directed, the subject involved (e.g., body weight, health, age, sex, etc.), and the preference and experience of the physician or veterinarian in charge.

Toxicity and therapeutic efficacy of the compositions of the invention can be determined by standard pharmaceutical procedures in experimental animals, e.g., by determining the LD50 (the dose lethal to 50% of the population) and the ED50 (the dose therapeutically effective in 50% of the population). The dose ratio between therapeutic and toxic effects is the therapeutic index and it can be expressed as the ratio ED50/LD50. Compositions that exhibit large therapeutic indices are preferred.

Suitable daily doses of memantine in therapeutic treatment of humans are about 0.01-10 mg/kg bodyweight on peroral administration and 0.001-10 mg/kg bodyweight on parenteral administration. For adults, suitable daily doses of memantine are within the range from about 5 mg to about 100 mg per day, preferably, from about 20 to about 40 mg per day. For pediatric subjects aged 4-14, it is preferred that memantine is administered as an oral, liquid dosage form, at about 0.5 mg/kg/day, up to a maximum dose of 10 mg/day. Titration to the maximum dose over about 4 weeks from a lower initial starting dose, e.g., about 2.5 mg/day, with weekly increases by about 2.5 mg/day, is highly recommended. For liquid, oral administration, memantine is dissolved in about one-half the liquid equivalent of the dose. For example, 10 mg memantine will be dissolved in 5 ml of the liquid formulation for administration.

Generally the amount of an atypical antipsychotic administered to a patient is an amount sufficient to have a therapeutic effect. In a preferred embodiment the amount of an atypical antipsychotic administered to a patient is an amount sufficient to treat at least one symptom or sign of schizophrenia, wherein the one sign or symptom may include, but are not limited to, delusions, hallucinations, disorganized speech (e.g., frequent derailment or incoherence), grossly disorganized or catatonic behavior and negative symptoms (e.g., affective flattening, alogia, avolition). One skilled in the art will recognize that the amount of atypical antipsychotic will vary with many factors including the potency of the atypical antipsychotic, the age and weight of the patient, and the severity of the condition or disorder to be treated. The dosages of the drugs used in the present invention must, in the final analysis, be set by the physician in charge of the case, using knowledge of the drugs, the properties of the drugs in combination as determined in clinical trials, and the characteristics of the patient, including diseases other than that for which the physician is treating the patient.

Non limiting daily dosage amounts for several atypical antipsychotics are provided herein; olanzapine, from about 0.25 to about 50 mg, preferably from about 1 to about 30 mg and most preferably from about 1 to about 25 mg; clozapine, from about 12.5 to about 900 mg, and more preferably from about 150 to about 450 mg; risperidone, from about 0.25 to about 16 mg daily and preferably from about 2 to about 8 mg daily; sertindole, from about 0.0001 to about 1.0 mg per kg; quetiapine, from about 1.0 to about 40 mg per kg; and ziprasidone, from about 5 to about 500 mg daily, and preferably from about 50 to about 100 mg.

Treatment duration can be short-term, e.g., several weeks (for example 8-14 weeks), or long-term until the attending physician deems further administration no longer is necessary.

### Administration

The present invention embodies the use of memantine as an adjunctive treatment to atypical antipsychotic in schizophrenia patients, wherein memantine may be administered alone or in combination with at least one atypical antipsychotic. In one embodiment memantine is administered in combination with at least one atypical antipsychotic in any manner which provides effective levels of the compounds in the body at the same time. In a preferred embodiment, both the atypical antipsychotic and memantine are administered orally.

Routes of administration other then oral are contemplated by the present invention, such as, transdermal, percutaneous, intravenous, intramuscular, intranasal or intrarectal routes of administration. In one embodiment, memantine may be administered by one route, such as oral, and the atypical antipsychotic may be administered by the transdermal, percutaneous, intravenous, intramuscular, intranasal or intrarectal route, in particular circumstances.

In one embodiment, the patient is an adult. In another embodiment, the patient is a pediatric patient.

Where memantine and at least one atypical antipsychotic are administered in combination, they may be administered as individual pharmaceutical compositions or in a unitary pharmaceutical composition. Where memantine and at least one atypical antipsychotic are administered as an unitary pharmaceutical composition, the dosage form may take any physical form which is pharmaceutically acceptable. In one embodiment unitary oral pharmaceutical compositions are particularly preferred. Such adjunctive pharmaceutical compositions contain an effective amount of each of the compounds, which effective amount is related to the daily dose of the compounds to be administered. Each adjunctive dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

### Definitions

For purposes of the present invention, “sustained release” or “modified release” means that the release of the therapeutically active agent occurs over an extended period of time leading to lower peak plasma concentrations (Cₘₐₓ) and a prolonged Tₘₐₓ as compared to “immediate release.” The “dissolution requirements” and “disintegration requirements” referred to above are conducted using the equipment and tests specified in the USP XXIV and conducted pursuant to the individual Official Monographs of USP XXIV (U.S. Pharmacopoeia and National Formulary, USP XXIV/NF 19, Chapter 1088, pages 2051-2056, 2000), incorporated herein by reference, for the particular therapeutically active agent(s) included in the tablet core.

A “therapeutically effective amount” means the amount of a compound that, when administered to a mammal for treating a state, disorder or condition is sufficient to effect such treatment. The “therapeutically effective amount” will vary depending on the compound, the disease and its severity and the age, weight, physical condition and responsiveness of the mammal to be treated. According to the instant invention, in one embodiment, a therapeutically effective amount of memantine is an amount effective to treat signs or symptoms of schizophrenia. The effective amount of the drug for pharmacological action, and therefore the tablet strength, depends on the disease itself, e.g., in schizophrenia, the patient is initially given a 5 mg dose and the dosage is progressively increased to 10 mg twice a day.

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

As used herein, the term “treat”, in all its verb forms, is used herein to mean to relieve or alleviate at least one sign or symptom of a disease in a subject, including for example, when the disorder is schizophrenia at least one sign or symptom may include, but is not limited to, delusions, hallucinations, disorganized speech (e.g., frequent derailment or incoherence), grossly disorganized or catatonic behavior and negative symptoms (e.g., affective flattening, alogia, avolition). The term “treat” may mean to relieve or alleviate the intensity and/or duration of a manifestation of disease experienced by a subject in response to a given stimulus (e.g., pressure, tissue injury, cold temperature, etc.). For example, in relation to dementia, the term “treat” may mean to relieve or alleviate cognitive impairment (including but not limited to impairment of memory and/or orientation) or impairment of global functioning (including but not limited to activities of daily living, ADL) and/or slow down or reverse the progressive deterioration in ADL or cognitive impairment. Within the meaning of the present invention, the term “treat” may also denote to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject. The term “treatment” means the act of “treating” as defined above.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value. For example, when referring to a period of time, e.g., hours, the present values (±20%) are more applicable. Thus, 6 hours can be, e.g., 4.8 hours, 5.5 hours, 6.5 hours, 7.2 hours, as well as the usual 6 hours.

“Memantine” refers to the free base 1-amino-3,5-dimethyladamantane or one of its pharmaceutically acceptable salts disclosed in U.S. Pat. Nos. 4,122,193, 4,273,774 and 5,061,703, all of which are hereby incorporated by reference. The term “salts” includes acid addition salts and addition salts of memantine.

The term “combination” as used herein refers to a pharmaceutical composition (formulation) comprising both memantine and at least one antipsychotic or two separate pharmaceutical compositions (formulations), each comprising a single drug of the invention (i.e., memantine or an antipsychotic), to be co-administered or administered conjointly.

Within the meaning of the present invention, the term “conjoint administration” or “co-administration” is used to refer to administration of memantine and at least one antipsychotic simultaneously in one composition, or simultaneously in different compositions, or sequentially. For the sequential administration to be considered “conjoint”, however, the memantine and at least one antipsychotic must be administered separated by a time interval that still permits the resultant beneficial effect for treating, preventing, arresting, delaying the onset of and/or reducing the risk of developing signs or symptoms associated with schizophrenia. For example, memantine and at least one antipsychotic are administered on the same day (e.g., each—once or twice daily), preferably within an hour of each other, and most preferably simultaneously.

The term “subject in need thereof” as used herein refers to a mammal. In particular, the term refers to humans diagnosed with schizophrenia and being treated with at least one atypical antipsychotic who are residually symptomatic.

The term “residually symptomatic” as used herein refers to a patient diagnosed with schizophrenia who are on a stable dose of antipsychotic but continue to demonstrate signs or symptoms of schizophrenia.

The term “schizophrenia” as used herein refers to a disorder that is at least partially due to one or more genetic mutations or polymorphisms in one or more genes involved in folate, cobalamin or pyridoxine metabolism in an individual that is schizophrenic and/or to one or more genetic mutations or polymorphisms in one or more genes involved in folate, cobalamin or pyridoxine metabolism in the mother of that individual. At present the nationally accepted definition for the diagnosis of schizophrenia is contained in Diagnostic and Statistical Manual for Mental Disorders, Fourth Edition, Washington, D.C (1994): American Psychiatric Association, hereby incorporated by reference in its entirety.

As used herein an individual is “schizophrenic” when the individual displays symptoms that would be accepted by an experienced psychiatrist to merit a diagnosis of schizophrenia. Such a diagnosis is based, at least in part, on the currently evolving guidelines for the diagnosis of schizophrenia which are listed in the successive editions of Diagnostic and Statistical Manual for Mental Disorders, put out by the American Psychiatric Association.

### EXAMPLES

The present invention will be better understood by reference to the following Examples, which are provided as exemplary of the invention, and not by way of limitation.

### Example 1

Overview of Study

This study is a multicenter, double-blind, placebo-controlled, parallel-group design, adjunctive study of memantine in schizophrenia patients who are on a stable dose of at least one atypical antipsychotic, but residually symptomatic.

The study consists of a 1-week screening period, followed by 8 weeks of double-blind treatment. This study involves a total of eight evaluations; screening, baseline, and at the end of weeks 1, 2, 3, 4, 6, and 8. Approximately 128 patients, 64 per treatment arm, are enrolled into the study.

The primary objective of the study is the change from baseline (visit 2) to Week 8 in PANSS total score. A further objective of the study is to further compare the efficacy of memantine as adjunctive treatment in Schizophrenia patients with persistent residual symptoms. The efficacy parameters for the secondary objective are CGI-S and CGI-I at week 8, the change from baseline to week 8 in the PANSS Positive Score, PANSS Negative Score, CDSS, BACS, and PANSS responders (a reduction of ≧10% in PANSS total score compared to baseline.

Patients

Patients in this study are in-patient or out-patient adults with schizophrenia who are on stable risperidone, olanzapine, quetiapine, aripiprazole, or ziprasidone treatment, but have persistent residual signs or symptoms of schizophrenia.

In order to be eligible to participate in the study, patients must meet the following criteria: (1) men and women 18 to 65 years of age at the time of screening visit; (2) diagnosis of schizophrenia (295.30 Paranoid Type, 295.10 Disorganized Type, 295.20 Catatonic Type, or 295.90 Undifferentiated Type) or schizoaffective disorder (295.70) as defined by DSM-IV based on the Structured Clinical Interview for DSM-IV (SCID); (3) schizophrenia or schizoaffective diagnosis for a minimum of 2 years; (4) Exhibits persistent positive symptoms as defined by both a BPRS total score of ≧26 and ≧4 on at least one item of the BPRS Psychosis Factor (conceptual disorganization, hallucinatory behavior, suspiciousness and unusual thought content (Items P2, P3, P6 and G9 from the PANSS) at both the screening and baseline visits; (5) positive symptoms have persisted for at least 3 months without exacerbation in the past 4 weeks; (6) on olanzapine, risperidone, quetiapine, aripiprazole, or ziprasidone monotherapy for at least 3 months before randomization, on a stable dose for at least 4 weeks before randomization, and willing to remain at that dose throughout the study; (7) if mood stabilizers or antidepressants are a part of the antipsychotic pharmacotherapy, must have been receiving each medication for at least 3 months before randomization, on a stable dose for at least 4 weeks before randomization, and willing to remain at that dose throughout the study; (8) women must be at least 2 years post-menopausal, surgically sterile, or practicing a medically acceptable method of contraception (rhythm and withdrawal methods are not acceptable); (9) women of childbearing potential must have a negative serum βHCG pregnancy test and agree to acceptable method of birth control (rhythm, withdrawal, barrier contraceptive methods, and abstinence are not acceptable); and (10) written informed consent by patient, guardian, or legally authorized representative (LAR).

Patients meeting any of the following criteria are to be excluded from the study: (1) patient's baseline total BPRS score changed 20% or more from the score at the screening visit; (2) primary or secondary psychiatric diagnosis of Bipolar I disorder, either manic or mixed episode, as defined by DSM-IV and based on the SCID; (3) patients who (as documented by informant's or in the investigator's opinion) have active suicide or homicide intent or a previous suicide or homicide attempt in the past 6 months; (4) patients with organic brain disease, dementia, or who have suffered a traumatic brain injury; (5) patients who in the Investigator's opinion might not be capable of completing the cognitive assessment; (6) patients with evidence or history of malignancy (other than excised basal-cell carcinoma) or any significant hematological, endocrine, cardiovascular, respiratory, renal, hepatic, or gastrointestinal disease. (If there is a history of such disease but the condition has been stable for more than 1 year and is judged by the Investigator not to interfere with the patient's participation in the study, the patient may be included, with the documented approval of the Study Physician); (7) patients who exhibit abnormalities on physical examination, or have abnormal vital signs, ECG, or clinical laboratory values unless these abnormalities are judged to be clinically insignificant as judged by the Investigator and the Study Physician; (8) history of substance dependence (including alcohol), excluding nicotine as defined by DSM-IV based on the SCID and relapse within the past 6 months or substance abuse within the past 3 months; (9) patients who test positive on a urine drug screen for drugs of abuse; (10) patients with known HIV infection; (11) female patients must not be lactating; (12) use of disallowed concomitant medication. (See List of Allowed/Disallowed Concomitant Medications in Appendix II); (13) patients who have been in a previous investigational study of memantine or neramexane; (14) patients who have received treatment with any investigational drug within 30 days or 5 half lives (whichever is longer) prior to study entry.; (15) patients with a history of hypersensitivity reaction to memantine or other drugs of the same class; and (16) patients who in the Investigator's opinion might not be suitable for other reasons.

Patients who meet all entry criteria at the screening and baseline visits are eligible to receive 8 weeks of double-blind treatment with memantine or placebo.

After the baseline visit, study visits are conducted at the end of weeks 1, 2, 3, 4, 6, and 8 (see Evaluations below).

Evaluation and Testing

The schedule of evaluation for each patient is as follows:

Initial screening visit consists of: (1) review study with patient and obtain written informed consent; (2) obtain medical, neurological, and psychiatric history; conduct PANSS Total, derive BPRS to determine disease severity; (3) perform physical examination; (4) record vital signs (including height and weight); (5) perform 12-lead ECG; (6) obtain blood and urine samples for laboratory determinations and βHCG pregnancy test in women of childbearing potential; (7) obtain urine sample for urine drug screen; (8) review concomitant medications; and (9) assess patient eligibility for enrollment via review of inclusion/exclusion criteria.

Baseline visit (Day=0) consists of: (1) review inclusion/exclusion criteria; (2) review study procedures with patient; (3) review concomitant medications; (4) review the occurrence of adverse events since the screening visit; (5) check vital signs; (6) conduct the following efficacy evaluations: PANSS total (derive BPRS), CGI-S, BACS, CDSS; (7) conduct the following EPS evaluations: Barnes, AIMS, and SAS scale; and (8) dispense medication as described below.

Assessment at day 7 consists of: (1) review concomitant medications; (2) review the occurrence of adverse events since the previous visit; (3) check vital signs; (4) conduct the following efficacy evaluation: PANSS total; and (5) dispense medication as described below.

Assessment at day 14 consists of: (1) review concomitant medications; (2) review the occurrence of adverse events since the previous visit; (3) check vital signs; (4) conduct the following efficacy evaluation: PANSS total; (5) obtain blood and urine samples for laboratory determinations; and (6) dispense medication as described below.

Assessment at day 21 consists of: (1) review concomitant medications; (2) review the occurrence of adverse events since the previous visit; (3) check vital signs; (4) conduct the following efficacy evaluation: PANSS total; and (5) dispense medication as described below.

Assessment at day 28 consists of: (1) review concomitant medications; (2) review the occurrence of adverse events since the previous visit; (3) check vital signs; (4) conduct the following efficacy evaluations: PANSS total, CGI-S, CGI-I, and BACS; (5) conduct the following EPS evaluations: Barnes, AIMS, and SAS scale; (6) obtain PK sample; (6) conduct urine pregnancy test; and (7) dispense medication as described below.

Assessment at day 42 consists of: (1) review concomitant medications; (2) review the occurrence of adverse events since the previous visit; (3) check vital signs; (4) conduct the following efficacy evaluations: PANSS total, CGI-S, CGI-I; and (6) dispense medication as described below.

Assessment at day 56 consists of: (1) review concomitant medications; (2) review the occurrence of adverse events since the previous visit; (3) check vital signs; (4) perform 12-lead ECG; (5) perform physical examination (including weight); (6) obtain blood and urine samples for laboratory determinations and βHCG pregnancy test; (7) obtain plasma sample for PK; (8) conduct the following efficacy evaluations: PANSS total, CGI-S, CGI-I, CDSS, and BACS; and (9) conduct the following EPS evaluations: Barnes, AIMS, and SAS scale.

Plasma samples taken during visits at end of weeks 4 and 8 for determination of memantine plasma concentrations following multiple dosing. Plasma samples are analyzed for memantine concentrations using a validated method. The plasma concentration-time profile of memantine in patients with schizophrenia is described using a mixed effects population model. Pharmacokinetic analyses is carried out using NONMEM® in order to estimate the pharmacokinetic parameters of memantine and intra- and inter-individual variability.

Efficacy is determined by performing a battery of tests as described below. The tests are conducted during patient visits as described above. Primary efficacy assessment is carried out using the Positive and Negative Symptom Scale—Total score (SCI-PANSS; Kay, et al. (1987) Positive and Negative Syndrome Scale (PANSS): Manual. New York: Multi-Health Systems, Inc.). The SCI-PANSS is rated based on a structured clinical interview with the patient and supporting clinical information obtained from family, hospital staff, or other reliable informants. Each item is scored on a 7-point (1-7) continuum and provides scores in nine clinical domains, including a positive syndrome, a negative syndrome, depression, a composite index, and general psychopathology. This scale can be conducted by an experienced clinician or other trained psychiatric rater with expertise in the assessment of patients with schizophrenia.

Secondary efficacy assessments are performed using one or more of the following exams:

Clinical Global Impression (CGI)—The 7-point severity scale (CGI-S) measures the overall severity of the illness in comparison to the severity of other patients the physician has observed and the 7-point improvement scale (CGI-I) measures the change from baseline in the overall severity of the illness for the individual patient. These assessments are made by the study physician.

PANSS Positive Score—This 7-item scale is derived from the PANSS. Each item—delusions, conceptual disorganization, hallucinations, excitement, grandiosity, suspiciousness, and hostility—is scored on a 7 point severity scale and is based on clinician observation.

PANSS Negative Score—This 7-item scale is derived from the PANSS, each item—blunted affect, emotional withdrawal, poor rapport, passive social withdrawal, difficulty in abstract thinking, lack of spontaneity, and stereotyped thinking—is scored on a 7 point severity scale and is based on clinician observation.

Calgary Depression Scale for Schizophrenia (CDSS; Addington D, Addington J, Atkinson M., Schizophrenia Research 19 (1996) 205-212), 9-item scale aids in the differentiation between symptoms of schizophrenia and depression. The semi-structured interview can be conducted by an experienced clinician and refers to symptoms from the past two weeks. Each item is rated on a 4-point scale, with 4 being the most severe and 1 being the absence of symptoms.

Brief Assessment of Cognition in Schizophrenia (BACS; Keefe 2002 NCDEU poster), measures treatment-related improvements in cognition and is specifically designed for use in schizophrenia. The BACS includes brief evaluations of: (1) verbal memory (list learning and recall); (2) working memory (digital sequencing task); (3) motor speed (placing items in a container); (4) semantic fluency (naming category instances); (5) letter fluency (controlled oral word association); (6) executive functions (Tower of London test); and (6) attention and motor speed (symbol coding). The scale can be performed by trained psychometricians.

Severity will be assessed using the Brief Psychiatric Rating Scale (BPRS; Overall, J. E. and Gorham, D. R. (1962) Psychol. Rep. 10:799-812.). The 18-item scale assesses both psychotic and non-psychotic symptom constructs on a 7 point severity scale. The severity ratings are based on patient self-report and on clinician observation during the interview. The BPRS score can be extracted from the PANSS total score, with a score range from 18 to 126. Items P2 through P7, N1, N2, and G1 through 10 of the PANSS constitute the BPRS total score and Items P2, P3, P6 and G9 constitute the BPRS Psychosis factor.

Administration of Memantine

Memantine hydrochloride 5 mg tablets (Commercially available from Forest Laboratories, Inc. under the tradename Namenda™) and matching placebo tablets are administered as film-coated tablets and dispensed at each clinic visit (e.g., visit 5 (Day 21), visit 6 (Day 28), etc.).

The study is conducted as a fixed-dose study in which all patients are titrated to the target dose of 20 mg/day. However, dosage adjustments are permitted for patients experiencing dose-limiting adverse events. Dose modifications should occur in 5 mg increments or decrements and the minimum dose for this study is 10 mg/day. The medical monitor must be made aware of any dosing modifications.

Patients who meet all of the eligibility criteria at visit 2 (baseline) are enrolled and dispensed study medication. Patients are dispensed ten 5 mg tablets of memantine hydrochloride and instructed to take one tablet daily. At visit 3 patients are dispensed twenty 5 mg tablets of memantine hydrochloride and instructed to take two tablets daily. At visits 4 and 5, patients are dispensed forty 5 mg tablets of memantine hydrochloride and instructed to take four tablets daily. At visits 6 and 7, patients are dispensed eighty 5 mg tablets of memantine hydrochloride s and instructed to continue taking four tablets daily. A diagrammatic representation of the titration and maintenance regimens is shown below in Table 1.

TABLE 1
Administration of Memantine

| | Titration | | | Maintenance |
|---|---|---|---|---|
| | Week 1 | Week 2 | Week 3 | Week 4-8 |
| Dose | 1 × 5 mg | 2 × 5 mg | 4 × 5 mg | 4 × 5 mg |

Efficacy

All efficacy analyses is based on the ITT Population. All statistical tests are two-sided hypothesis tests performed at the 5% level of significance.

Primary analyses is performed on the ITT Population using the Last Observation Carried Forward (LOCF) approach at Week 8. In these analyses, the last post-baseline observed value before the missing value is carried forward to impute the missing value. The observed cases (OC) approach is used for supportive analyses, where only the observed values at each visit are used for analyses. The LOCF approach is used at each visit for supportive analyses.

For the change from baseline to Week 8 in the PANSS total scores, the comparison between memantine and placebo is performed using two-way analysis of covariance (ANCOVA) with treatment group and center as the factors and the baseline scores as a covariate. Descriptive statistics are calculated by visit.

The secondary efficacy parameters are: Change from baseline (visit 2) in CGI-S; Change from baseline (visit 2) in the PANSS Positive Score; Change from baseline (visit 2) in the PANSS Negative Score; Change from baseline (visit 2) in CDSS; CGI-I; Change from baseline (visit 2) in BACS; and PANSS responders (10% in PANSS total score compared to baseline).

The CGI-I and PANSS responders are analyzed using the CMH test, controlling for study center. For change from baseline in CGI-S, PANSS Positive Score, PANSS Negative Score, CDSS, and BACS, the comparison between memantine and placebo is performed using a two-way ANCOVA with treatment group and study center as factors and the baseline scores as a covariate. Descriptive statistics are presented by visit and treatment group.

Subjects are examined for improvements in secondary endpoints, i.e., improvements in the behavior and symptomology, compared to placebo-treated individuals. Patients are examined for improvements in, for example, one or more of the following: improvements in delusions, hallucinations, disorganized speech (e.g., frequent derailment or incoherence), grossly disorganized or catatonic behavior and negative symptoms (e.g., affective flattening, alogia, avolition).

Improvements may be determined, for example, on the diagnostic scale, PANSS-Total Score. Similarly, improvements may be determined on secondary scales such as the CGI-S, CGI-I, PANSS-Positive, PANSS-Negative, CDSS, and BACS, as compared to placebo treatments.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. A method for treating schizophrenia in a patient in need thereof, the method comprising administering to the patient a therapeutically effective amount of memantine, or a pharmaceutically acceptable salt thereof, and a therapeutically effective amount of at least one atypical antipsychotic.

2. The method of claim 1, wherein the treatment comprises administering memantine, or a pharmaceutically acceptable salt thereof, as an adjunctive treatment where the patient is being treated with at least one atypical antipsychotic.

3. The method of claim 1, wherein the memantine, or a pharmaceutically acceptable salt thereof, and the atypical antipsychotic are co-administered.

4. The method of claim 3, wherein the memantine, or a pharmaceutically acceptable salt thereof, and the atypical antipsychotic are co-administered as an unitary dosage form.

5. The method of claim 1, wherein the patient is residually symptomatic.

6. The method of claim 1, wherein the patient is being treated with an atypical antipsychotic selected from the group consisting of olanzapine, clozapine, risperidone, sertindole, quetiapine, ziprasidone, surmontill and aripiprazole.

7. The method of claim 1, wherein the memantine, or a pharmaceutically acceptable salt thereof, is administered in a range of from about 2.5 mg to about 100 mg/day.

8. The method of claim 7, wherein the memantine, or a pharmaceutically acceptable salt thereof, is administered in a range of from about 5 to about 80 mg/day.

9. The method of claim 8, wherein the memantine, or a pharmaceutically acceptable salt thereof, is administered at about 5 mg/day.

10. The method of claim 8, wherein the memantine, or a pharmaceutically acceptable salt thereof, is administered at about 10 mg/day.

11. The method of claim 8, wherein the memantine, or a pharmaceutically acceptable salt thereof, is administered at about 20 mg/day.

12. The method of claim 1, wherein the memantine, or a pharmaceutically acceptable salt thereof, is administered once a day or twice a day (b.i.d.).

13. The method of claim 1, wherein the memantine, or a pharmaceutically acceptable salt thereof, is administered as a modified release formulation.

14. The method of claim 1, wherein the memantine, or a pharmaceutically acceptable salt thereof, is administered as an immediate release formulation.

15. The method of claim 1, wherein the memantine, or a pharmaceutically acceptable salt thereof, is administered in tablet form.

16. The method of claim 1, wherein the memantine, or a pharmaceutically acceptable salt thereof, is administered as a pharmaceutical formulation comprising a plurality of beads.

17. The method of claim 16, wherein the pharmaceutical formulation comprises immediate release beads, or modified release beads, or a combination thereof.

18. The method of claim 1, wherein the memantine, or a pharmaceutically acceptable salt thereof, is administered in liquid form.

19. A method for treating at least one sign or symptom of schizophrenia in a patient in need thereof, the method comprising administering to the patient a therapeutically effective amount of memantine, or a pharmaceutically acceptable salt thereof, and a therapeutically effective amount of at least one atypical antipsychotic, wherein the sign or symptom is selected from the group consisting of delusions, hallucinations, disorganized speech, catatonic behavior, affective flattening, alogia and avolition.

20. A method for treating schizophrenia in patient in need thereof comprising administering to the patient a therapeutically effective amount of memantine, or a pharmaceutically acceptable salt thereof and a therapeutically effective amount of a typical antipsychotic.
